(19) United States
(12) Patent Application Publication
(10) Pub. No.: US 2006/0004249 A1
(43) Pub. Date: Jan. 5, 2006

(54) Systems and methods for sizing cardiac assist device

(75) Inventors: Stephanie Kute, Stewartsville, NJ (US); Steven Zung-Hong Wu, Hillsborough, NJ (US)

Correspondence Address: Scully, Scott, Murphy & Presser, 400 Garden City Plaza, Garden City, NY, 11530, US

(73) Assignee: Ethicon Incorporated, Somerville, NJ (US)

(21) Appl. No.: 10/881,511

(22) Filed: Jun. 30, 2004

(51) Int. Cl. A61F 13/00; A61B 17/08

(52) U.S. Cl. 600/37; 606/151

(57) ABSTRACT

Systems and methods for sizing a cardiac assist device to a heart. A bio-compatible material placed as a cardiac assist device over portions of the heart as desired, is sized to fit the exterior contours of the heart by gathering excess material using a reduction ring, draw strings, or a roller. The remaining material of the cardiac assist device may be sutured, clipped, or otherwise anchored to achieve the desired fit of the cardiac assist device to the heart. Subsequent adjustments to increase or decrease the size of the cardiac assist device may be made to accommodate subsequent changes to the size of the heart by re-performing the reduction ring, draw strings, or roller procedure and re-orienting the sutures, clips, or other anchoring techniques to fit the cardiac assist device to the subsequent heart size.

### BACKGROUND OF THE INVENTION

1. Field of the Invention

The invention generally relates to systems and methods for sizing a cardiac assist device to fit over portions of a heart. More specifically, the invention relates to systems and methods for sizing a chronic heart failure passive restraint device to fit over portions of a heart, while permitting subsequent adjustments of the device in order to accommodate changes in the size of the heart.

2. Prior Art

Heart failure syndrome is a highly debilitating and degenerative disorder resulting from damage to the heart muscle. The damage to the heart muscle may be caused by a number of conditions, including coronary artery disease, long standing hypertension, leaky heart valve(s), and infections.

Heart failure typically occurs when a weakened heart cannot pump an adequate amount of blood to meet the demands of the body's other organs and tissues. The defining characteristic in the progression of heart failure is that there is eventually a reduction of the heart's ability to meet the metabolic needs of the body.

Whatever the cause or source of damage, the heart's ability to pump adequate amounts of blood to support the body's needs is diminished, and the progressive deterioration of cardiac physiology and function occurs. The inadequate supply of oxygen-rich blood often causes people with heart failure to experience shortness of breath and fatigue during even routine daily activities. As the condition progresses, the contraction rate of the heart increases in response to the decreasing cardiac output. As a result, the chambers of the heart, particularly the ventricles of the heart, become increasingly enlarged as the heart tries to compensate for its inefficiencies. FIGS. 1a-1c show representative stages of progressive deterioration of a heart, wherein FIG. 1a shows a normal heart H with appropriately sized atrial chambers 1 and 2, and appropriately sized ventricular chambers 3 and 4. FIG. 1b shows slightly enlarged ventricular chambers 3 and 4, and FIG. 1c shows increasingly enlarged ventricular chambers 3 and 4. Ultimately, a complex process of damaging structural and functional changes to the heart results. Ventricular dilation results in thinning of the ventricular wall, which elevates the wall stress. This increase in wall stress leads to altered gene expression at the cellular level that results in attenuated adrenergic response, impaired myocyte function, cardiomyocyte hypertrophy, altered extracellular matrix production and cell death. This remodeling process continues as the body tries to continually compensate for ineffective pumping and eventually leads to heart failure.

The disease of heart failure is common, lethal, and expensive to treat. An estimated 5.1 million Americans have heart failure with approximately 500,000 new cases diagnosed each year. In 1999, an estimated $20.3 billion in directs costs were spent for the care of heart failure patients. Heart failure is also the most common cause of hospitalization for patients 65 years and older in the United States. The mortality rate is 50% at five years for patients diagnosed with heart failure, and to date, there are limited treatment alternatives available.

Certain cardiac disease treatment devices have been proposed to help alleviate the disease of heart failure. For example, U.S. Pat. No. 6,425,856 provides a cardiac constraint device comprised of a jacket made of biologically compatible material. FIG. 1d illustrates how the jacket 20 may be positioned around the heart H to improve cardiac function. The jacket surrounds a valvular annulus of the heart and at least the ventricular lower extremities of the heart. Once positioned as desired around the heart, the jacket 20 is sutured to the heart. A piezoelectric material or separately provided stays with receptacles are then used to decrease the volume of the jacket to fit more closely over the heart.

The jacket 20 thus works on a passive, mechanical level to reduce periodic myocardial over-stretch and wall stress, and serves as a constant “reminder” to the heart of how the heart should perform. The jacket thus encourages down-regulation of increased local neurohormonal activity, and reduces or eliminates cardiomyocyte maladaptive gene expression. As a result, the jacket may slow or halt the progressive deterioration of the heart and may stimulate reverse remodeling of the heart.

Suturing the jacket to the heart is a cumbersome procedure however, and the stays are prone to loosening from their receptacles. Even where U-clips or staples have been used in place of sutures, the procedures of fitting the jacket around the heart and placing the U-clips to secure the jacket to the heart the procedure are time-consuming and cumbersome. Further, subsequent adjustments to the jacket, where provided for, may accommodate for decreases in the size of the heart but may not account for increases in the heart size. As a result, precise positioning of the jacket around the heart has proved time consuming and maintaining the jacket closely over the heart over time has been problematic. Further still, positioning and securing the jacket about the heart using endoscopic tools and techniques as the heart is beating has proved challenging.

An alternative procedure for surrounding a heart with a cardiac assist device endoscopically places and manipulates a bio-compatible mesh sheet wrapping means around the heart as described in co-pending U.S. patent application Ser. No. ______, filed ______, 2004, (Attorney Docket No. 17386) of common assignment herewith, the entire disclosure of which is incorporated herein by reference. The wrapping means is secured directly to the heart by sutures or to anchoring devices separately secured to the heart. This co-pending application however, does not provide the various sizing or subsequent adjustment systems and methods of the present invention as described further hereinbelow.

In view of the above, a need exists for systems and methods that provide more reliable sizing of a cardiac assist device to fit closely about a heart. A further need exists for systems and methods that permit subsequent adjustments to maintain the cardiac assist device in a close-fitting position around the heart.

### SUMMARY OF THE INVENTION

The systems and methods of the invention provide for sizing a cardiac assist device to fit closely adjacent portions of a heart. The cardiac assist device may help reduce distention of chambers of the heart and may encourage remodeling of a damaged or diseased heart. The cardiac assist device is comprised of a bio-compatible material having an open first end with a seam about its periphery and a second end opposite the first end. In some embodiments of the invention, the second end is closed to form a sack-like cardiac assist device into which the heart is placed. In other embodiments of the invention, the second end is open to form a tubular-like cardiac assist device into which the heart is placed.

According to the systems and methods of the invention, one or more openings, are provided through the chest wall of a being. The cardiac assist device, instruments, and visualization means are inserted into a chest cavity of the being through the one or more openings in order to place, fit and secure the cardiac assist device to the heart. The visualization means may be an endoscope, a fiber optic cable or a camera on an elongate member, for example, permitting a medical professional to view the various procedures performed as they occur according to the invention.

In practice, once the cardiac assist device is in the chest cavity of the being, the heart is received through the open first end of the cardiac assist device until an apical region of the heart approaches the second end thereof. The first end is then sutured, or otherwise secured, to a base of the heart or to anchoring devices that are secured to the heart, such as those described in co-pending U.S. patent application Serial No. ______, (Attorney Docket No. 17386) the entire disclosure of which has been incorporated herein by reference.

After securing the first end to the heart, excess material of the cardiac assist device is then gathered to conform the cardiac assist device to the heart. The gathered material is then secured, thereby fitting the cardiac assist device more closely to the heart. When desired, subsequent adjustments to the cardiac assist device may be performed to increase or decrease the size of the cardiac assist device by gathering or releasing the excess material, as appropriate, thereby promoting an ongoing close fit of the cardiac assist device with the heart.

According to the systems and methods of the invention a material gathering device is used to gather the excess material of the cardiac assist device. One embodiment of the material gathering device comprises drawstrings incorporated into and weaved throughout the bio-compatible material. The drawstrings are endoscopically drawn or released, as needed, and then secured, in order to fit the cardiac assist device closely to the heart. Paired ends of each drawstring are manipulated to secure the drawstrings and cardiac assist device in a desired position around the heart. Each drawstring may be secured, for example, by one of tying, snapping, clipping, zipping, twisting or cable-tying a respective set of paired ends to one another. Subsequent adjustments to the cardiac assist device in order to fit an increased or decreased heart size are readily achieved by unsecuring the drawstrings, re-drawing or releasing the drawstrings to accommodate the changed heart size, and then re-securing the paired ends of each drawstring to fit the cardiac assist device closely to the heart.

According to another embodiment of the systems and methods described herein, the material gathering device is a separately provided reduction ring. The reduction ring is slid over the material at the second end of the cardiac assist device to gather excess material extending therefrom. The gathered material is then secured to maintain the cardiac assist device closely to the heart. The reduction ring is then removed. Subsequent adjustments to the cardiac assist device in order to accommodate changing heart sizes are readily accomplished by un-securing the gathered material, re-performing the reduction ring procedure, and re-securing the gathered material in a desired position so that the cardiac assist device again fits closely to the heart. In some embodiments the ring at the distal end of a rod is positioned over one end of the cardiac assist device to loosely gather the excess material. In other embodiments, the ring is positioned over on end of the cardiac assist device and a slidable rod is maneuvered to close the ring more snugly around the excess material.

In yet another embodiment of the systems and methods described herein, an anterior seam is provided on the material of the cardiac assist device. The anterior seam is located between the first end and the second end of the material. The anterior seam is provided with closure devices on either side of the anterior seam. The closure devices are endoscopically aligned with and connected to one another to close the anterior seam of the cardiac assist device. A separately provided adjustment roller attaches to the closed anterior seam and closure devices to gather excess material of the cardiac assist device and fit the device more closely to the heart. The excess material is gathered by rotating the roller in one direction and then securing the gathered material. The adjustment roller is then removed from the chest cavity, or the roller remains in place and a handle of the adjustment roller is detached from the roller and removed from the chest cavity. In either case, the adjustment roller readily provides subsequent adjustments to the cardiac assist device by un-securing the gathered material, re-performing the adjustment roller procedure to gather excess material to accommodate the changed heart size, and re-securing the gathered material.

Of course, the artisan should readily appreciate that the various material gathering devices and techniques described herein may also be used in combination in order to fit the cardiac assist device even more precisely to the heart.

The various embodiments of the system and methods of the invention described herein provide several advantages over and solve many of the problems posed by prior known devices and methods of sizing a cardiac assist device to a heart. The various exemplary embodiments of the invention minimize the time and cumbersome efforts previously required to fit and suture a cardiac assist device to the heart. The various exemplary embodiments of the invention also provide increased flexibility in terms of adjusting the cardiac assist device to accommodate increased or decreased sizes in the heart after initial placement of the device over the heart. Further, the various embodiments of the invention simplify the endoscopic placing and securing of the cardiac assist device about the heart even during beating heart procedures and may be used in non-endoscopic, i.e. open chest, or hybrid procedures. Moreover, the various embodiments of the invention described herein promote a more precise fit of the cardiac assist device to the heart, even as remodeling of the heart may occur.

The above and other features of the invention, including various novel details of construction and combinations of parts, will now be more particularly described with reference to the accompanying drawings and claims. It will be understood that the various exemplary embodiments of the invention described herein are shown by way of illustration only and not as a limitation thereof. The principles and features of this invention may be employed in various alternative embodiments without departing from the scope of the invention.

### BRIEF DESCRIPTION OF THE DRAWINGS

These and other features, aspects, and advantages of the apparatus and methods of the present invention will become better understood with regard to the following description, appended claims, and accompanying drawings where:

FIGS. 1a-c illustrate progressive states of deterioration of a heart representative of heart failure conditions.

FIG. 1d illustrates a prior art mesh jacket placed around a heart.

FIG. 2 illustrates a cardiac assist device having drawstrings according to a first embodiment of the invention.

FIGS. 3a-3f illustrate various securing means at paired ends of a drawstring according to the cardiac assist device shown in FIG. 2

FIG. 4 illustrates the cardiac assist device of FIG. 2 placed about a heart according to the invention.

FIGS. 5a and 5b illustrate alternative cardiac assist devices without drawstrings according to a second embodiment of the invention.

FIG. 6 illustrates a reduction ring according to the invention.

FIG. 7 illustrates the cardiac assist device of FIG. 5 placed about the heart using the reduction ring of FIG. 6 according to a second embodiment of the invention.

FIG. 8 illustrates material of the cardiac assist device gathered by the reduction ring of FIG. 6 according to the invention.

FIGS. 9a-9c illustrate another embodiment of a reduction ring according to the invention.

FIG. 10 illustrates a means of securing the excess material gathered by the reduction ring of FIG. 6 according to the invention.

FIG. 11 illustrates a cardiac assist device having an anterior seal with closure devices according to a third embodiment of the invention, wherein the anterior seal is in an open position.

FIG. 12 illustrates the cardiac assist device of FIG. 11, wherein the anterior seal is closed.

FIG. 13 illustrates the ports and instruments used to place the cardiac assist device of FIG. 11 with an open anterior seam within the chest cavity of a being.

FIG. 14 illustrates the ports and instrument used to place the cardiac assist device of FIG. 11 with a closed anterior seam within the chest cavity of a being.

FIG. 15a illustrates one embodiment of a reduction roller according to the invention.

FIG. 15b illustrates an alternative embodiment of a reduction roller having a detachable shaft according to the invention.

FIGS. 16a-16c illustrate various stages of fitting the cardiac assist device of FIGS. 11-13 to a heart using the reduction roller of FIG. 15 in accordance with the invention.

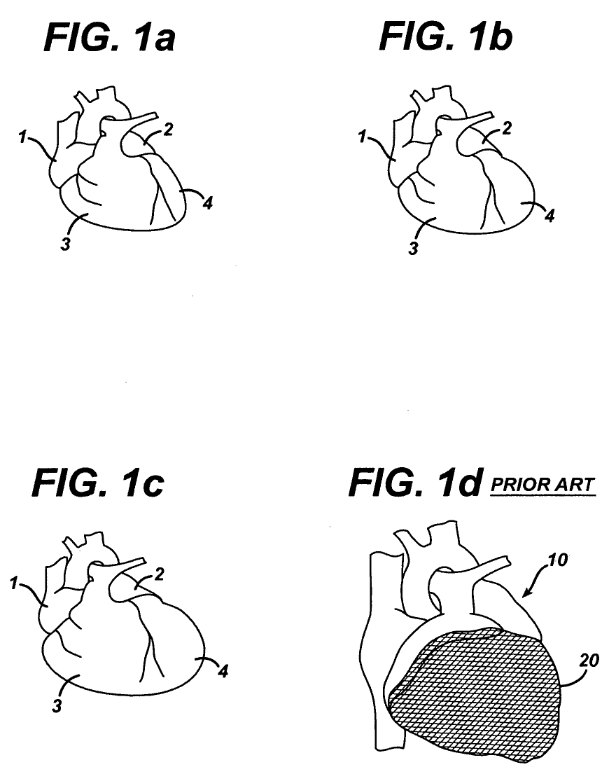

### DETAILED DESCRIPTION OF THE INVENTION

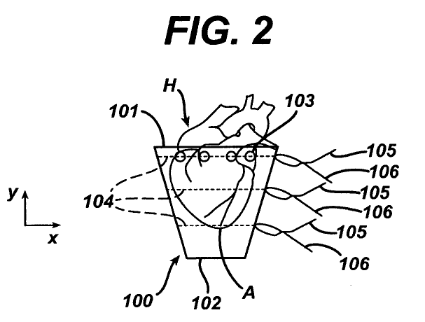

FIG. 2 illustrates a cardiac assist device 100 according to one embodiment of the the systems and methods of the invention. More specifically FIG. 2 illustrates an exemplary cardiac assist device 100 that surrounds the heart H. The device 100 is comprised of a sack-like bio-compatible material having an open first end 101 and a closed second end 102 opposite the first end. The first end includes a seam along a periphery of the material. The material comprising the cardiac assist device may be a polymeric, a metal, a plastic, an elastic, or other material, such as Merselene or Prolene mesh, known in the art suitable for surrounding the heart and reducing the propensity of chambers of the heart to distend. The material may be woven or non-woven, and does not have to be meshed.

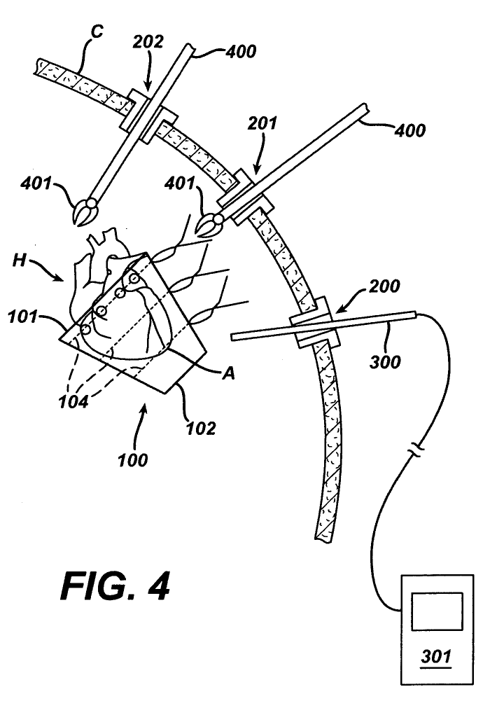

FIG. 2 further illustrates drawstrings 104 (shown in dashed lines) incorporated into and weaved through the material of the cardiac assist device. The artisan should appreciate that the drawstrings could instead be disposed in a pocket formed in the material of the cardiac assist device. The drawstrings 104 are used as a material management means, for example, to gather excess material and secure the cardiac assist device to the heart in a desired position about the heart, as discussed in greater detail below with respect to FIG. 4.

The drawstrings 104 can be comprised of suture-like material, such as, for example, Merselene, Prolene, stainless steel, c-flex, or other suitable material known in the art. The skilled artisan should appreciate that any number of drawstrings may be used, although three drawstrings are shown in FIG. 2 for illustrative purposes. Likewise, the artisan should appreciate that the placement and orientation of the drawstrings may vary from that shown in FIG. 2, such that more vertically angled drawstrings, or other orientations, may be used in place of, or in combination with, the generally horizontally parallel oriented drawstrings of the cardiac assist device shown in FIG. 2. Each drawstring 104 includes a pair of ends 105, 106 which extend from the material of the device 100. Each pair of ends provides a means of tensioning the drawstrings to gather the excess material of the cardiac assist device. Each pair of ends further provides a means of securing the drawstring in place once the desired fit of the cardiac assist device adjacent to the heart is achieved.

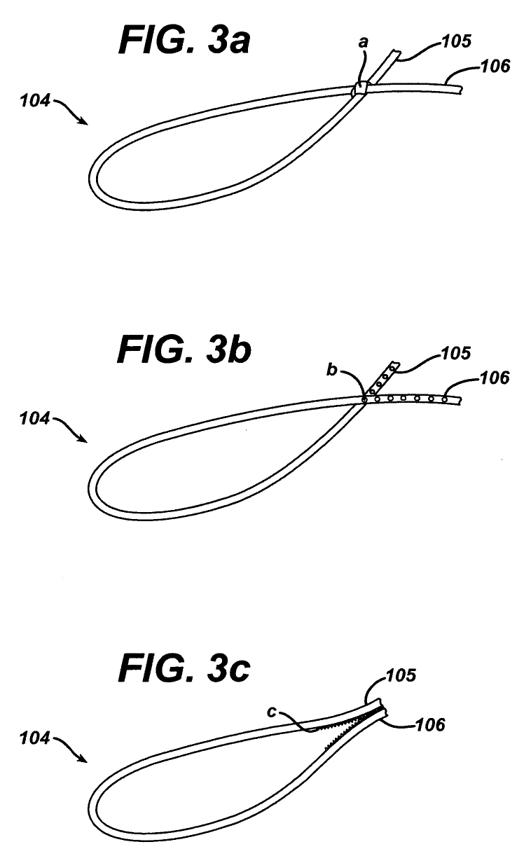

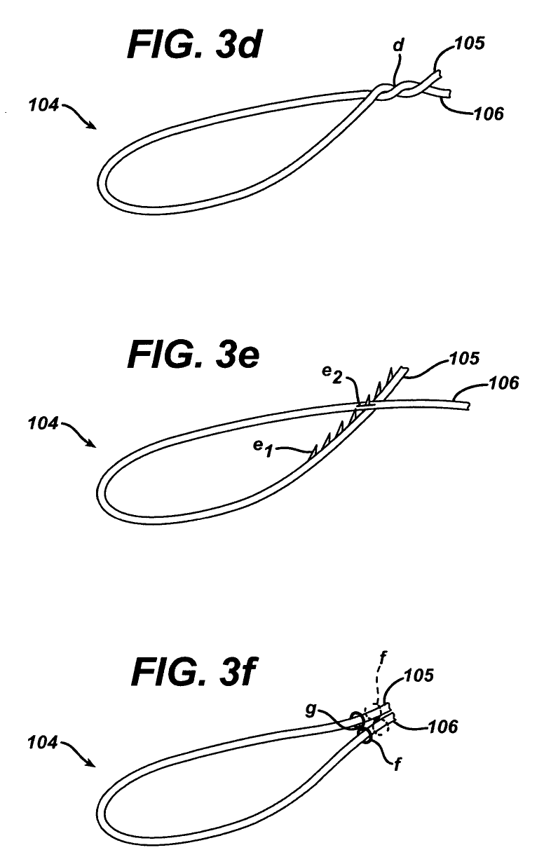

FIGS. 3a-3e illustrate various alternatives for the pair of ends 105, 106 used for securing the drawstrings 104 to one another. For example, FIG. 3a illustrates ends 105, 106 that are tied to one another forming a knot (a) to secure the drawstring as desired. FIG. 3b illustrates ends 105, 106 having snaps (b) that are snapped together to secure the drawstrings to one another. FIG. 3c illustrates ends 105, 106 having teeth (c) that are zipped to secure the drawstrings to one another. FIG. 3d illustrates ends 105, 106 that are twisted (d) to secure the drawstrings to one another. FIG. 3e illustrates ends 105, 106 having protrusions (e1) and an opening (e2) that are interlocked to cable-tie and secure the drawstrings to one another. FIG. 3f illustrates ends 105, 106 that are clipped to secure the drawstrings to one another. The clip may be a sliding clip (f) that opens and closes via hinge (g) to position and secure the clip (f) at a desired position along the drawstrings. An open position of clip (f) is shown in dashed lines in FIG. 3f. The respective paired ends 105, 106 of drawstrings 104 thus pose a relatively simple method of securing the drawstrings to one another once tensioned to fit the cardiac assist device closely to the heart.

In practice, as illustrated in FIG. 4, one or more openings, such as a plurality of ports 200-202, are provided through the chest wall C. The ports 200-202, or other openings, are provided in a conventional manner known in the art and permit access to the chest cavity of a patient or subject. For example, trocar tubes may be used for one or more of the ports 200-202. Alternatively, the one or more openings can be an opening within the chest wall or below the chest wall (subyxphoid). In either case, the opening may be held open with tape or other known retraction device. A visualizing means, such as an endoscope 300, a fiber-optic cable or a camera on an elongated member, is placed into the chest cavity through one of the openings or ports 200-202, and preferably through the lowest port 200. The endoscope, for example, permits a medical professional to view devices and instruments placed in the chest cavity and medical procedures as they occur in the chest cavity. A monitor 301, for example, external to the patient or subject connects to the endoscope 300 and provides views of the devices, instruments and procedures on the interior of the chest cavity.

The cardiac assist device 100 is placed in the chest cavity through one of the openings or ports, and preferably through one of upper ports 201, 202. In this manner, conventional instruments 400, such as graspers, inserted through a respective upper port 201, 202 are able to grasp and manipulate the cardiac assist device to a desired position about the heart. Each of instruments 400 can have grasping elements 401 at a distal end thereof, whereas proximal ends of each instrument 400 extend external to the chest cavity for manipulation of the instrument 400 and activation of the grasping elements 401 by the medical professional. The manipulation and activation of the instruments and grasping elements is done under observation using the endoscope 300, or other visualizing means, according to the invention.

After the cardiac assist device 100 is placed in the chest cavity, the instruments 400 grasp the first end 101 of the cardiac assist device and pull the device over the heart until the apex, or apical region, A of the heart approaches the second end 102 of the device. Thereafter, the cardiac assist device is conformed to the shape of the heart and secured to the heart by tensioning the drawstrings 104 and securing the paired ends 105, 106 of each drawstring, respectively, to one another. The manipulation of the drawstrings and paired ends thereof are also done using the instruments 400, or other grasping device known in the art.

If desired, the cardiac assist device may be further secured directly or indirectly to the heart by sutures, U-clips, staples, adhesives or other securing means, including the anchoring devices of co-pending U.S. patent application Ser. No. ______, (Attorney Docket No. 17386) referenced above. The instruments 400 may be used to perform the suturing, for example, or to otherwise secure the cardiac assist device to the heart or anchoring device. Where anchoring devices, such as those in co-pending U.S. patent application Ser. No. ______, (Attorney Docket No. 17386) are used, and the cardiac assist device is secured to the anchoring devices, it may be preferable to place the anchoring devices on the heart, using instruments 400, prior to placing the cardiac assist device over the heart. In this manner, the cardiac assist device can be simply and quickly secured to the anchoring devices once the cardiac assist device is positioned as desired adjacent the heart.

Once the cardiac assist device is in place about the heart as desired, the proximal ends of the instruments 400 are manipulated by the medical professional to grasp and pull, or release, the drawstrings 104 in order to conform the material of the cardiac assist device closely to the heart. The instruments 400 are then further manipulated to tie, or otherwise secure, the drawstrings in order to maintain the cardiac assist device in close conformity with the heart.

The relatively simple securing devices and methods used according to this embodiment of the invention, permits subsequent adjustments of the cardiac assist device 100 to be readily achieved in a minimally invasive manner after cardiac function has been evaluated. Such adjustments could be done endoscopically, for example, or non-endoscopically, after the patient has stabilized from the initial surgery and placement of the cardiac assist device. Such adjustments could be achieved, for example, using similar openings and instruments as discussed above with respect to the initial placement of the device. The subsequent adjustment would be achieved, for example, using instruments 400 to un-secure the drawstrings and pull, or release, the drawstrings to accommodate the adjusted heart size. Instruments 400 would then be manipulated, as before, to secure the drawstrings to accommodate the changed heart size. Such subsequent adjustments may help eliminate the plateau effect some patients experience after initial placement of the cardiac assist device.

Because relatively few surgical instruments are required to place and secure the exemplary cardiac assist device of FIG. 2 over the heart, the medical professional is better able to fit the cardiac assist device to the heart in an uncluttered surgical atmosphere. At the same time, the medical professional is able to view the procedures of placing and securing the cardiac assist device to the heart resulting in easier and more precise sizing of the cardiac assist device to the heart even as the heart beats. As a result, smaller ventricular volume and a more pointed apex of the heart is more likely to be achieved due to the systems and methods herein described.

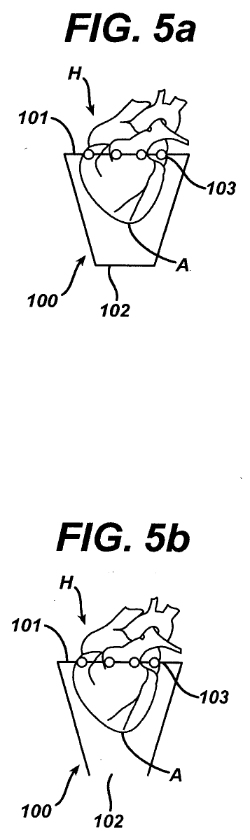

FIGS. 5a-10 illustrate another embodiment of systems and methods for sizing a cardiac assist device to a heart, wherein like numerals are used to refer to like components. The cardiac assist device 100 in FIGS. 5a and 5b generally corresponds to the device 100 shown in FIG. 2 except that the drawstrings 104 of FIG. 2 are omitted in the device 100 of FIGS. 5a and 5b. Like that of FIG. 2, the cardiac assist device 100 of FIG. 5 is comprised of similar material having an open first end 101 having a seam along the periphery thereof, and a second end 102 opposite the first end. The material is as described above with reference to FIG. 2. The second end 102 may be closed to form a sack-like cardiac assist device as in FIG. 5a, or may be open to form a tubular-like cardiac assist device as in FIG. 5b.

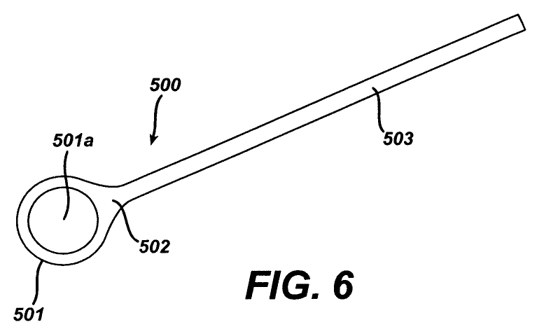

FIG. 6 illustrates one embodiment of a reduction ring instrument 500 used, as a material management means, to gather excess material of the cardiac assist device 100 of either of FIGS. 5a and 5b after the device 100 has been placed about the heart of a patient or subject. The reduction ring instrument is comprised of a ring 501 at a distal end of the reduction ring instrument 500, a shaft 502 extending from the ring 501, and a rod 503 extending from the shaft 502. The rod 503 comprises a proximal portion of the reduction ring instrument that can be manipulated by the medical professional during endoscopic procedures, for example.

The ring 501 is comprised of soft compliant material, such as C-Flex, for example, or other material rendering the open loop 501a of the ring 501 adjustable to tighten around excess material of the cardiac assist device. The shaft 502 is comprised of a malleable material, such as a malleable 316 stainless steel permitting the ring 501 to be placed at various angles as desired, and the rod 503 is comprised of a rigid material. The shaft 502 should be sufficiently malleable to allow re-shaping or re-orienting of the ring during endoscopic procedures.

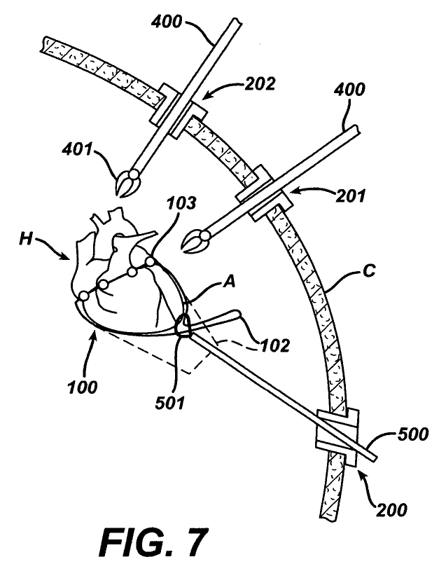

Referring now to FIG. 7 wherein the sack-like cardiac assist device 100 of FIG. 5a is shown by way of illustration, though similar procedures as described herein are understood to apply equally to the tubular-like cardiac assist device of FIG. 5b as well. In FIG. 7, for example, the cardiac assist device 100 is placed into the chest cavity of a patient or subject through one of the openings or plurality of ports 200-201 extending through the chest wall C, in conventional manner and similar to that described with reference to FIG. 4 above. Instruments 400 are likewise inserted into the chest cavity, preferably through upper openings or ports 201, 202. Although not shown in FIG. 7, a visualizing means such as endoscope 300 (FIG. 4) is inserted through lower port 200, for example, enabling the medical professional to view the devices, instruments and procedures in the chest cavity as they occur. As before the visualization means may also be a fiber optic cable or camera on an elongate member. Proximal portions of the instruments 400 and endoscope 300 extend externally from the chest cavity and are available to the medical professional for manipulation as the various procedures occur in the chest cavity.

The instruments 400 are used to grasp and manipulate the cardiac assist device 100 over the heart H until an apical region A of the heart approaches the second end 102 of the cardiac assist device. Thereafter, the first end 101 of the cardiac assist device is secured directly or indirectly to the base of the heart using sutures, u-clips, staples, adhesives or other securing devices such as anchoring device as disclosed in co-pending U.S. patent application Ser. No. ______, (Attorney Docket No. 17386) referenced above.

The endoscope 300, or other visualizing means, is then preferably transferred to one of upper ports 201, 202 and the reduction ring instrument 500 is inserted into the port 200 vacated by the endoscope. The instruments 400 may remain in the openings or ports 201, 202 even as the endoscope is inserted therein. Of course, either or both of the instruments 400 may be removed from the openings or ports 201, 202 at the discretion of the medical professional until needed again. The reduction ring instrument 500 is then manipulated to gather excess material within an opening 501a at the closed end 102 of the cardiac assist device near the apical region of the heart.

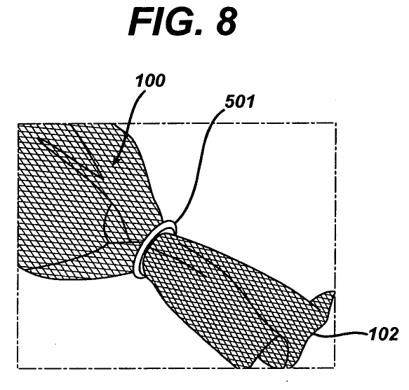

FIG. 8 illustrates a view of excess material gathered at the second end 102 of a cardiac assist device 100 according to the systems and methods of the invention. In the embodiment shown in FIG. 8, the ring 501 of the reduction ring instrument is shown surrounding the excess material of a tubular-like cardiac assist device 100, for example.

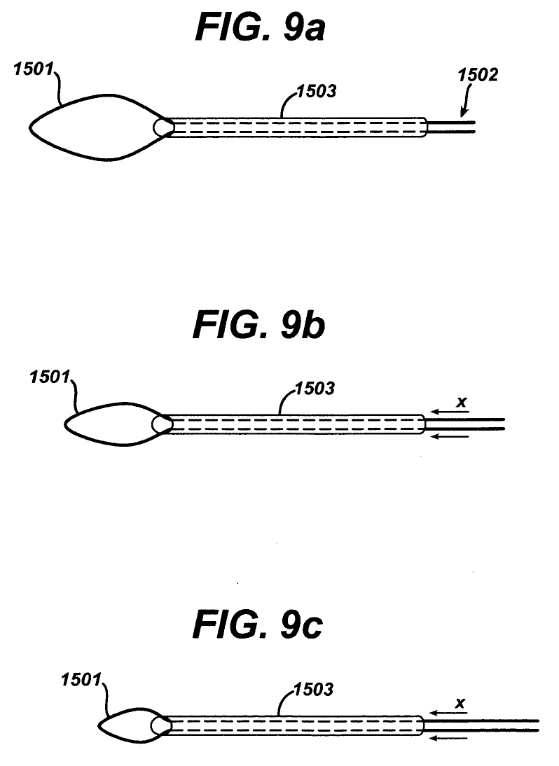

FIGS. 9a-c shows another embodiment of the reduction ring 1500 comprised of a ring 1501 protruding from a distal end of a hollow slidable rod 1503. The ring 1501 is comprised of a bio-compatible, suture-like material, for example, as known in the art. Free ends 1502 of the material extend through a proximal end of the slidable rod 1503. The ring 1501 is placed around excess material at one end of the cardiac assist device as described with reference to FIGS. 7 and 8. Thereafter, as shown in FIGS. 9b-c, the slidable rod 1503 is moved towards the ring (arrows x) rendering the ring smaller to more snugly fit around the excess material gathered therein.

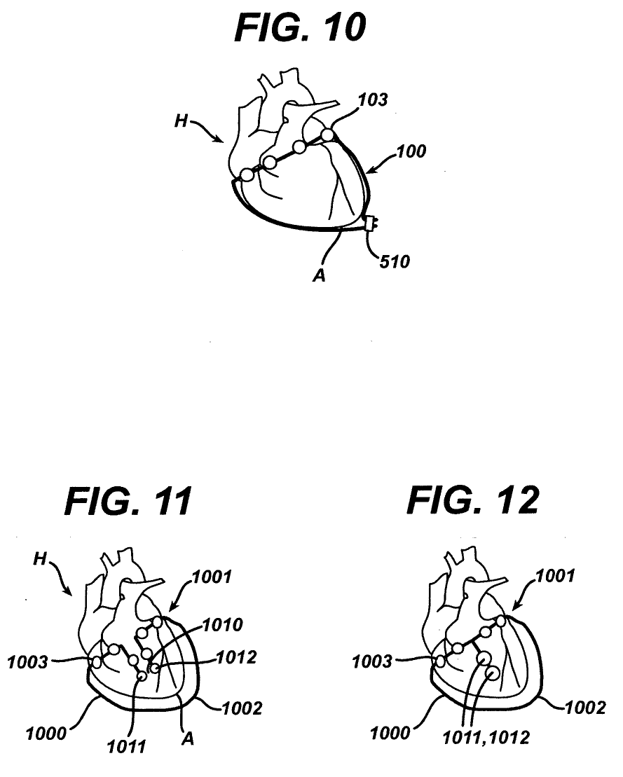

As shown in FIG. 10, the gathered material using either embodiments of the reduction ring, is then either secured by known securing means, such as clips 510, or other securing means known in the art, such as sutures, in order to maintain the cardiac assist device in close conformity to the heart. Some or all of the excess material extending beyond the clips 510, for example, may be removed at the discretion of the medical professional, such as with endoscopic cutting instruments known in the art. Retaining some of the excess material beyond the clip permits greater flexibility to accommodate future increases in the heart size subsequent to the initial placement of the cardiac assist device.

Subsequent adjustments to the cardiac assist device using the reduction ring techniques and devices described above are readily available to accommodate changing heart sizes. Where a decreased heart size occurs, for example, the reduction ring 501 or 1501 is simply re-applied to gather any additional excess material. The securing means, or clips 510, are then re-positioned to accommodate the new heart size. Where an increased heart size occurs, then the clips 510 are removed to free the excess material, the reduction ring is re-applied to gather any new amount of excess material, and the clips 510 are re-applied to accommodate the increased heart size. If the securing means is re-usable, i.e., can be opened and closed, then the same securing means can simply be slid towards or away from the heart in order to adjust for decreased or increased heart sizes, respectively.

As in the exemplary embodiments described above with reference to FIGS. 2-4, the embodiments of the reduction ring systems and methods described with reference to FIGS. 5a-10 provide relatively easy sizing of the cardiac assist device in an uncluttered operating space. Re-shaping of the heart resulting in the preferred more pointed apex of the heart is thus more readily promoted. Further, the initial placement and subsequent adjustment procedures, if desired, may be performed endoscopically, or non-endoscopically, as well.

FIGS. 11-14c illustrate a third exemplary embodiment of the cardiac assist device according to the systems and methods of the invention. Similar to the earlier described cardiac assist devices, the cardiac assist device 1000 shown in FIG. 11, for example, is also comprised of a bio-compatible material. As before, the material is as described above with reference to FIG. 2.

As shown in FIG. 11, the cardiac assist device 1000 is pouch-shaped and oversized relative to the heart H. The pouch-shaped device has an open first end 1001 with a seam along the periphery thereof, and a closed second end 1002, the second end being opposite the first end. When placed around the heart H, the closed second end surrounds an apical region A of the heart, and the first end is secured to a base of the heart. As in other embodiments of the invention described above, securing means 1003, such as sutures, clips, staples, adhesives, drawstrings as described herein, magnets, anchoring devices as described in co-pending U.S. patent application Ser. No. ______, (Attorney Docket No. 17386), or other securing means known in the art are used to secure the first end of the cardiac assist device directly or indirectly to the heart initially.

Referring still to FIG. 11, the cardiac assist device further comprises an anterior seam 1010 extending between the first end 1001 and second end 1002 of the device. More specifically, the anterior seam 1010, shown open in FIG. 11, extends from the first end 1001 towards, but not as far as, the second end 1002 of the cardiac assist device. Closure devices and corresponding receiving members 1011, 1012 are placed on either side of the anterior seam 1010. The closure devices and receiving members 1011, 1012 may be snaps, buttons, magnets, or other fastening means known in the art.

FIG. 12 illustrates the closure devices 1011, 1012 connected to one another in order to close the anterior seam. The anterior seam is generally closed once the cardiac assist device has been placed adjacent the heart in a desired position.

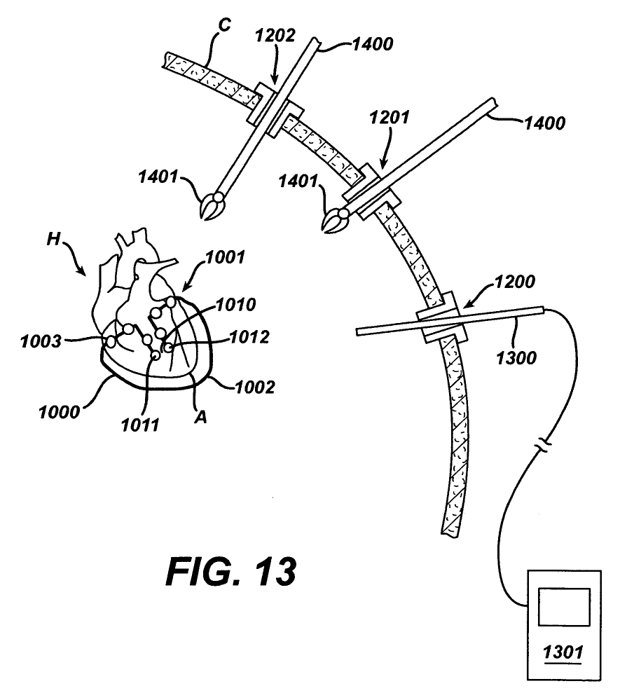

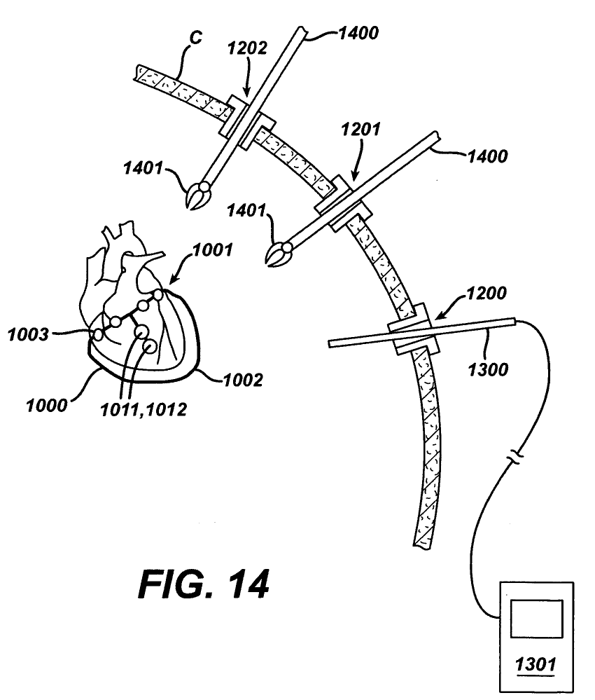

In practice, with reference first to FIG. 13 and FIG. 14, a plurality of ports 1200-1202, or other openings, are provided through the chest wall C. As in the earlier described embodiments, the ports 1200-1202, or other openings, are provided in a conventional manner known in the art and permit access to the chest cavity of a patient or subject. The openings may alternatively be an opening within the chest wall or below the chest wall (subyxphoid) of the patient. In either case the opening may be held open using tape or other retraction device. A visualizing means 1300, such as an endoscope, a fiberoptic cable, or camera on an elongate member, is placed into the chest cavity through one of the openings or ports, and preferably initially through the lower opening or port 1200. The cardiac assist device 1000 is placed into the chest cavity through one of the remaining openings or ports 1201, 1202, for example.

Conventional instruments 1400 are inserted into the chest cavity through the openings or ports 1201, 1202. Similar to the instruments 400 in earlier embodiments, each instrument 1400 has grasping elements 1401 at a distal end thereof. Each instrument 1401 also has a proximal end extending external to the chest cavity. The visualizing means 1300 likewise has a proximal end extending externally from the chest wall. In this manner, the proximal ends of visualizing means 1300 and instruments 1400 are available for manipulation by a medical professional. At the same time, the visualizing means 1300 permits the medical professional to observe the various devices, instruments and procedures within the chest cavity as they occur.

After initially placing the cardiac assist device 1000 into the chest cavity through one of the openings or ports, the anterior seam 1010 of the cardiac assist device is open, as shown in FIG. 13. The instruments 1400 then grasp the first end of the cardiac assist device 1000 and maneuver the cardiac assist device to a desired position around the heart. Thereafter, as shown in FIG. 14, the instruments 1400 are further manipulated to close the anterior seam 1010 by aligning and securing the closure devices 1011 on one side of the anterior seam with the closure devices 1012 on the other side of the anterior seam. In this manner, the cardiac assist device loosely surrounds the heart but does not reduce ventricular volume.

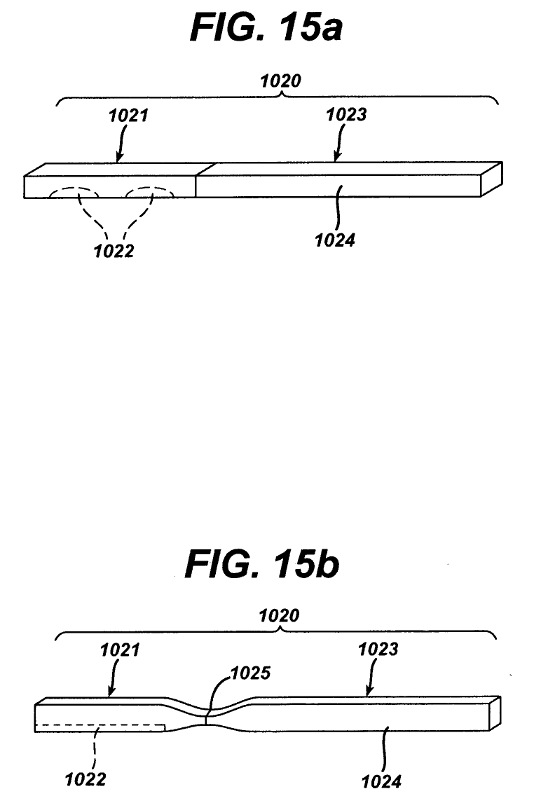

FIG. 15a shows a reduction roller 1020, as a material management means, that releasably attaches to the closed anterior seam and closure devices. The roller 1020 in FIG. 15a includes a distal end 1021 having attachment means 1022, and a proximal end 1023 including a handle or shaft 1024. The attachment means 1022 in FIG. 15a a are sockets, for example, that fit over the exposed heads of closure devices 1011, 1012 when the anterior seam is closed. The attachment means 1022 in FIG. 15a could instead be magnets, Velcro, or other attachment means known in the art that would similarly connect to the heads of closure devices 1011, 1012 when the anterior seam is closed. The reduction roller is preferably thin and composed of metal, or other strong, bio-compatible polymer. The shaft 1024 is stainless steel.

FIG. 15b shows an alternative reduction roller 1020, as a material management means, having a handle 1024 that detaches from the distal end 1021 of the roller via coupling 1025. FIG. 15b further shows an alternative attachment means 1022 comprised of rails, for example, that slide over the closed anterior seam to connect to the cardiac assist device. The attachment means 1022 may thus comprise sockets, rails, magnets, or other attachment means known in the art suitable to attach to the closure devices and anterior seam of the cardiac assist device.

In practice, the reduction roller 1020 is inserted into the chest cavity of a being through any one of the openings or ports 1200-1202 as described above with reference to FIGS. 13 and 14, even as the instruments 1400 or visualizing means 1300 continue to reside in the openings or ports. Of course, the instruments 1400 or visualizing means 1300 may be repositioned or removed from any of the openings or ports in order to better accommodate the reduction roller in the discretion of the medical professional.

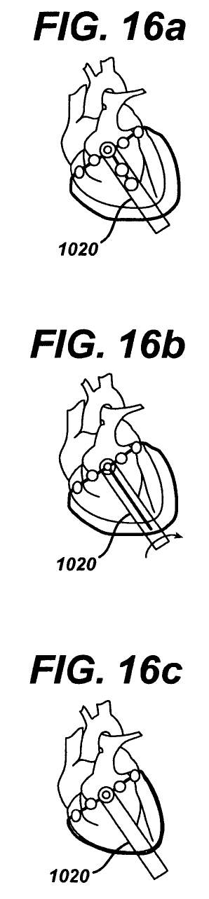

FIGS. 16a-16c, wherein the chest wall, ports, instruments and visualizing means are omitted from illustration but understood to exist as shown and described earlier with respect to FIGS. 11-15b, show various stages of fitting the cardiac assist device 1000 to the heart using the reduction roller of FIG. 15a, for example. More specifically, FIG. 16a shows the attachment means 1022 of the roller 1020 grasping, or otherwise releasably attached to, the closure devices 1011, 1012 and closed anterior seam 1010. FIG. 16b then shows the roller 1020 lifted by the medical professional to pull the excess material of the cardiac assist device 1000 to a more taut position. Referring still to FIG. 16b, the roller 1020 is then rotated to wrap the excess material around the roller. As a result, as shown in FIG. 16c, the material of the cardiac assist device fits closely to the contours of the heart. The excess material gathered on the roller is then secured with sutures, clips, staples, or other suitable anchoring devices as in earlier described embodiments including those in co-pending U.S. patent application Ser. No. ______, (Attorney Docket No. 17386). The roller 1020 is then pulled out from the gathered material, or left in place while the shaft 1022 is released from the roller. The artisan should appreciate that were the reduction roller of FIG. 15b used instead, the handle 1024 could simply be removed by uncoupling the handle 1024 from the attachment means 1022 of the roller using coupling 1025. In this maner, the distal end 1021 of the roller would remain with the cardiac assist device as it is secured in place about the heart.

If necessary, additional sizing of the cardiac assist device at the apical region of the heart may be accomplished using the reduction ring technique described above. Alternatively, if additional sizing of the device at the apical region of the heart is necessary, the drawstring technique described above can be used to tighten and more precisely fit the device to the apex of the heart. In this latter case, the pouch shaped cardiac assist device would be provided with at least one drawstring near the apical region of the heart.

Subsequent adjustments to the cardiac assist device using the roller technique are readily available to accommodate changing heart sizes. Where either a decreased or increased heart size occurs, the gathered material is unsecured and unraveled, the roller with the handle is re-applied to the closed anterior seam, and the roller is rotated to gather any additional excess material. The gathered material is then re-secured at the new position in order to accommodate the new heart size. As before, the roller is then either removed, or the handle only is removed, and the gathered material is secured in a desired position about the heart. If desired, the reduction ring or drawstring techniques may also be used to even more precisely fit the apical region of the heart to the cardiac assist device, as discussed above.

As in the exemplary embodiments described above with reference to FIGS. 2-10, the exemplary embodiments described with reference to FIGS. 11-16c, the reduction roller systems and methods provide relatively easy sizing of the cardiac assist device in a relatively uncluttered operating space. Re-shaping of the heart resulting in the preferred more pointed apex of the heart is thus more easily promoted. The initial placement and adjustments of the cardiac assist device may be performed endoscopically or non-endoscopically as well.

The various exemplary embodiments of the invention as described hereinabove do not limit different embodiments of the present invention. The bio-compatible material, described herein as an exemplary cardiac assist device, is not limited to the materials, designs, or shapes referenced herein for illustrative purposes only, and may comprise various other materials, designs or shapes suitable for the procedures described herein as should be appreciated by one of ordinary skill in the art. For example, the material may be a fabric like that described in U.S. Pat. No. 6,682,476 or materials such as those described in U.S. Pat. No. 6,595,912, which are incorporated herein by reference.

While there has been shown and described what is considered to be preferred embodiments of the invention, it will, of course, be understood that various modifications and changes in form or detail could readily be made without departing from the spirit or scope of the invention. For example, while the invention has been described with reference to an endoscopic procedure, the system described herein can also be used in non-endoscopic, open chest or hybrid procedures that would not rely solely upon openings in a patient's chest. It is therefore intended that the invention be not limited to the exact forms described and illustrated herein, but should be constructed to cover all modifications that may fall within the scope of the appended claims.

What is claimed is:

1. An adjustable cardiac assist device for receiving at least a portion of a heart, the device comprising:
a bio-compatible material having a first end and a second end opposite the first end, wherein the at least a portion of a heart is received within the material between the first and second ends; and
at least one drawstring incorporated into the material for conforming the material to a shape of the at least a portion of the heart.

2. The cardiac assist device of claim 1, wherein the heart is received within the material such that a base of the heart approaches the first end, and an apical region of the heart approaches the second end thereof.

3. The cardiac assist device of claim 1, wherein the at least one drawstring further comprises a material management means for gathering and releasing material according to the size of the heart.

4. The cardiac assist device of claim 1, wherein the first end is open having a seam along a periphery of the open first end, and the second end is closed, the material thereby forming a pouch for receiving the heart.

5. The cardiac assist device of claim 1, wherein the first end is open having a seam along a periphery of the open first end, and the second end is open, the material thereby forming a tube for receiving the heart.

6. The cardiac assist device of claim 3, wherein the at least one drawstring comprises two or more drawstrings, each drawstring fitting different portions of the material to a corresponding portion of the heart.

7. The cardiac assist device of claim 6, wherein each of the at least one drawstring further comprises a set of paired ends, each set of paired ends having means to secure the at least one drawstring at a desired position adjacent the heart.

8. The cardiac assist device of claim 7, wherein each set of paired ends further comprises one or more of corresponding ties, snaps, clips, teeth, twists or cable-ties as the means for securing each set of the paired ends together.

9. An adjustable cardiac assist device for receiving at least a portion of a heart, the device comprising:
a bio-compatible material having a first end and a second end opposite the first end, wherein the at least a portion of a heart is received within the material between the first and second ends; and
an annular ring for gathering excess material to fit the material closely adjacent the heart.

10. The cardiac assist device of claim 9, wherein the heart is received within the material such that a base of the heart approaches the first end, and an apical region of the heart approaches the second end thereof.

11. The cardiac assist device of claim 9, wherein the first end is open having a seam along a periphery of the open first end, and the second end is closed, the material thereby forming a pouch for receiving the heart.

12. The cardiac assist device of claim 9, wherein the first end is open having a seam along a periphery of the open first end, and the second end is open, the material thereby forming a tube for receiving the heart.

13. The cardiac assist device of claim 9, wherein the annular ring forms a material management means comprising:
the annular ring at a distal end of the material management means
a shaft extending from the annular ring, and a rod extends from the shaft to form a proximal end of the material management means.

14. The cardiac assist device of claim 13, wherein the annular ring is further comprised of a soft, compliant material, the shaft is comprised of a malleable material permitting positioning of the annular ring at various angles, and the rod is comprised of a rigid material.

15. The cardiac assist device of claim 12, further comprising a securing device to secure the material gathered to fit the cardiac assist device to the heart in a desired position.

16. The cardiac assist device of claim 11, wherein a securing device is elected from a group consisting of a clip, suture, tie and staple secures the gathered material.

17. The cardiac assist device of claim 13, wherein the annular ring forms a material management means comprising:
a hollow slidable rod having a distal end and a proximal end;
flexible material passing through said rod and forming the annular ring at the distal end of the rod and having free ends extending from the proximal end of the rod, whereby the rod is slidable to adjust the size of the annular ring.

18. The cardiac assist device of claim 17, wherein the material of the annular ring is a suture material.

19. An adjustable cardiac assist device for receiving at least a portion of a heart, the device comprising:
a bio-compatible material having a first end and a second end opposite the first end, wherein the at least a portion of a heart is received within the material between the first and second ends; and
an anterior seam between the first end and the second end; and
closure devices on either side of the anterior seam.

20. The cardiac assist device of claim 19, wherein the closure devices are at least one of snaps, buttons, magnets on one side of the anterior seam and corresponding receiving members on an opposite side of the anterior seam.

21. The cardiac assist device of claim 19, further comprising:
a reduction roller material management means for attaching to the anterior seam and closure devices and gathering or releasing excess material to conform the cardiac assist device to the heart.

22. The cardiac assist device of claim 21, wherein the reduction roller further comprises a distal end having attachment means, and a proximal end having a handle, the attachment means attaching to the anterior seam and closure devices, to gather excess material and conform the cardiac assist device to the heart.

23. The cardiac assist device of claim 22, wherein the attachment means further comprises one of sockets, magnets, and rails attaching to at least one of the closure devices and anterior seam.

24. The cardiac assist device of claim 23, wherein the reduction roller is comprised of a bio-compatible polymer, metal, or combinations thereof, and the handle is comprised of stainless steel.

25. The cardiac assist device of claim 24, further comprising a means for securing the material gathered by the reduction roller.

26. The cardiac assist device of claim 25, wherein the means for securing the gathered material is elected from a group consisting of a clip, suture, tie, staple and anchoring device.

27. A method for sizing a cardiac assist device on a heart, the method comprising:
providing one or more openings through a chest wall and into a chest cavity of a patient;
inserting a visualizing means into the chest cavity through one of the one or more openings;
inserting a cardiac assist device into the chest cavity through one of the one or more openings;
inserting at least one instrument into the chest cavity through a one of the one or more openings, each instrument having a distal end within the chest cavity for manipulating the cardiac assist device and a proximal end extending externally from the chest cavity for manipulation by a medical professional;
conforming the cardiac assist device to at least a portion of the heart; and
securing the cardiac assist device in the conformed position adjacent the heart.

28. The method of claim 27, wherein providing the cardiac assist device comprises providing a bio-compatible material having a first end and a second end opposite the first end, wherein at least a portion of the heart is received between the first and second ends.

29. The method of claim 28, wherein conforming the cardiac assist device further comprises gathering or releasing the material using a material management means to conform the material of the cardiac assist device to the at least a portion of the heart.

30. The method of claim 29, wherein the material management means is comprised of at least one drawstring incorporated throughout the material the at least one drawstring being tightened or released to accommodate the heart.

31. The method of claim 30, wherein the at least one drawstring comprises two or more drawstrings, each drawstring fitting different portions of the material to a corresponding portion of the heart.

32. The method of claim 31, wherein the at least one drawstring includes a set of paired ends, the set of paired ends having means to secure the at least one drawstring at a desired position about the heart.

33. The method of claim 32, wherein the means to secure the at least one drawstring comprises one of tying, snapping, clipping, twisting, zipping, or cable-tying each set of paired ends together.

34. The method of claim 29, wherein the material management means is further comprised of an annular ring for gathering excess material to fit the cardiac assist device in conformity with the at least a portion of the heart.

35. The method of claim 34, wherein the material of the cardiac assist device is conformed to the at least a portion of the heart by sliding the annular ring over the cardiac assist device to gather excess material and fit the cardiac assist device adjacent the heart.

36. The method of claim 35, wherein after sliding the annular ring over the cardiac assist device, the annular ring is tightened about the gathered materials.

37. The method of claim 36, whereby tightening the annular ring is achieved by sliding a hollow rod towards the annular ring.

38. The method of claim 37, wherein the gathered material is secured to maintain the fit of the cardiac assist device in the conformed position about the heart by one of tying, clipping, suturing and stapling.

39. The method of claim 29, wherein inserting the cardiac assist device further comprises inserting a cardiac assist device having an anterior seam extending from the first end towards the second end of the cardiac assist device.

40. The method of claim 39, wherein the cardiac assist device further comprises closure devices on either side of the anterior seam that close the anterior seam by one of snapping, buttoning, or magnetically attaching to one another.

41. The method of claim 40, wherein the at least one instrument manipulates the cardiac assist device within the chest cavity such that a base of the heart is positioned near the first end, and an apical region of the heart is positioned near the second end of the cardiac assist device and the anterior seam is open initially.

42. The method of claim 41, further comprising manipulating the at least one instrument to close the anterior seam by aligning and securing the closure devices to one another.

43. The method of claim 42, wherein the material management means further comprises a reduction roller that gathers or releases excess material to conform material of the cardiac assist device to at least a portion of the heart.

44. The method of claim 43, wherein the reduction roller further comprises a distal end having attachment means, and a proximal end having a handle, the attachment means attaching to the closure devices and closed anterior seam within the chest cavity and the handle extending externally from the chest cavity for manipulation by the medical professional to conform and fit the cardiac assist device to the at least a portion of the heart.

45. The method of claim 44, wherein the reduction roller is lifted and rotated to gather excess material of the cardiac assist device.

46. The method of claim 45, wherein the gathered material is secured using one of clips, sutures, ties, anchoring devices or staples by further manipulation of the at least one instrument.

47. The method of claim 46, wherein the handle is detached after the material is gathered and secured to conform and fit the cardiac assist device to the heart.

48. The method of claim 29, wherein subsequent adjustments to conform the material of the cardiac assist device to at least portions of the heart are achieved by un-securing the gathered material, manipulating the material management means to re-gather excess material to conform the material to a new position about the at least a portion of the heart, and re-securing the gathered material to fit the material of the cardiac assist device in the new position about the heart.

49. The method of claim 48, wherein the at least one instrument is further manipulated to secure the first end of the cardiac assist device directly to one of the heart or anchoring devices separately secured to the heart.

50. The method of claim 49, wherein the first end is secured to one of the heart or anchoring devices using one of sutures, clips, and staples.
